(19) United States
(12) Patent Application Publication
(10) Pub. No.: US 2019/0110831 A1
(43) Pub. Date: Apr. 18, 2019

(54) TREATMENT TOOL

(71) Applicant: OLYMPUS CORPORATION, Tokyo (JP)

(72) Inventor: Koichi KUDO, Tokyo (JP)

(73) Assignee: OLYMPUS CORPORATION, Tokyo (JP)

(21) Appl. No.: 16/215,714

(22) Filed: Dec. 11, 2018

Related U.S. Application Data
(63) Continuation of application No. PCT/JP2016/068428, filed on Jun. 21, 2016.

(51) Int. Cl. A61B 18/08

(52) U.S. Cl. CPC A61B 18/085; A61B 2018/00095

(57) ABSTRACT

A treatment tool includes: a pair of openable/closable jaws; a substrate provided on at least one of the jaws; a wiring pattern provided on one surface of the substrate in a state of being electrically insulated from the substrate, the wiring pattern including an electric resistance pattern configured to generate heat when a current is carried to the electric resistance pattern, and a connecting section configured to be electrically continuous to the electric resistance pattern; and a heat transfer plate disposed facing the one surface and configured to transfer, to a living tissue, the heat in contact with the living tissue; and a lead wire configured to be a current-carrying path to the wiring pattern, the lead wire being positioned on a side separated from the heat transfer plate with respect to the wiring pattern and being electrically connected to the connecting section from the separated side.

### CROSS-REFERENCE TO RELATED APPLICATION

This application is a continuation of PCT international application Ser. No. PCT/JP2016/068428 filed on Jun. 21, 2016 which designates the United States, incorporated herein by reference.

### BACKGROUND

The present disclosure relates to a treatment tool.

In the related art, a treatment tool that treats a living tissue (examples of the treatment including bonding (or anastomosis) and dissection) by applying energy to the living tissue is known (see, for example, JP 2012-24583 A).

The treatment tool disclosed in JP 2012-24583 A is provided with a pair of living tissue-gripping jaws. Each of the pair of jaws is provided with an energy applying structure generating thermal energy and applying the thermal energy to a living tissue.

For example, it is conceivable that a heater, a heat transfer plate, an adhesive sheet, and a lead wire to be described below constitute the energy applying structure in the interest of thinning.

The heater is provided with a substrate and a wiring pattern provided on one surface of the substrate. Further, the wiring pattern is provided with an electric resistance pattern generating heat when a current is carried thereto and a connecting section electrically continuous to the electric resistance pattern.

A conductor such as copper constitutes the heat transfer plate. The heat transfer plate is disposed facing one surface of the substrate (surface where the wiring pattern is provided) and transfers heat from the electric resistance pattern to a living tissue (applies thermal energy to a living tissue).

The adhesive sheet is interposed between the heater and the heat transfer plate for adhesive fixing between the heater and the heat transfer plate.

Here, the heater is longer than the heat transfer plate. The heater has one end side (side where the connecting section is provided) protruding from the heat transfer plate when assembled. Further, the lead wire as a current-carrying path to the electric resistance pattern is connected to the connecting section provided on that side. In other words, thinning of the energy applying structure may be achieved by the lead wire being positioned on one surface of the substrate (side where the heat transfer plate is disposed).

### SUMMARY

There is a need for a treatment tool with which the diameter of a living tissue-gripping structure may be reduced.

A treatment tool according to one aspect of the present disclosure includes: a pair of openable/closable jaws; a substrate provided on at least one of the pair of jaws; a wiring pattern provided on one surface of the substrate in a state of being electrically insulated from the substrate, the wiring pattern including an electric resistance pattern configured to generate heat when a current is carried to the electric resistance pattern, and a connecting section configured to be electrically continuous to the electric resistance pattern; and a heat transfer plate disposed facing the one surface and configured to transfer, to a living tissue, the heat from the electric resistance pattern in contact with the living tissue; and a lead wire configured to be a current-carrying path to the wiring pattern, the lead wire being positioned on a side separated from the heat transfer plate with respect to the wiring pattern and being electrically connected to the connecting section from the separated side.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating a treatment system according to a first embodiment;

FIG. 2 is a diagram illustrating the distal end part of a treatment tool;

FIG. 3 is a diagram illustrating the distal end part of the treatment tool;

FIG. 4 is a diagram illustrating a first energy applying structure;

FIG. 5 is a diagram illustrating the first energy applying structure;

FIG. 6 is a diagram for describing an opening/closing operation of first and second jaws;

FIG. 7 is a diagram for describing the opening/closing operation of the first and second jaws;

FIG. 8 is a diagram illustrating a heater according to a second embodiment;

FIG. 9 is a diagram illustrating the heater according to the second embodiment;

FIG. 10 is a diagram illustrating a heater according to a third embodiment;

FIG. 11 is a diagram illustrating the heater according to the third embodiment; and

FIG. 12 is a diagram illustrating a treatment tool according to a fourth embodiment.

### DETAILED DESCRIPTION

Hereinafter, modes for carrying out the present disclosure (hereinafter, embodiments) will be described with reference to accompanying drawings. It should be noted that the present disclosure is not limited by the embodiments described below. Further, in the description of the drawings, the same reference numerals are used to designate the same parts.

### First Embodiment

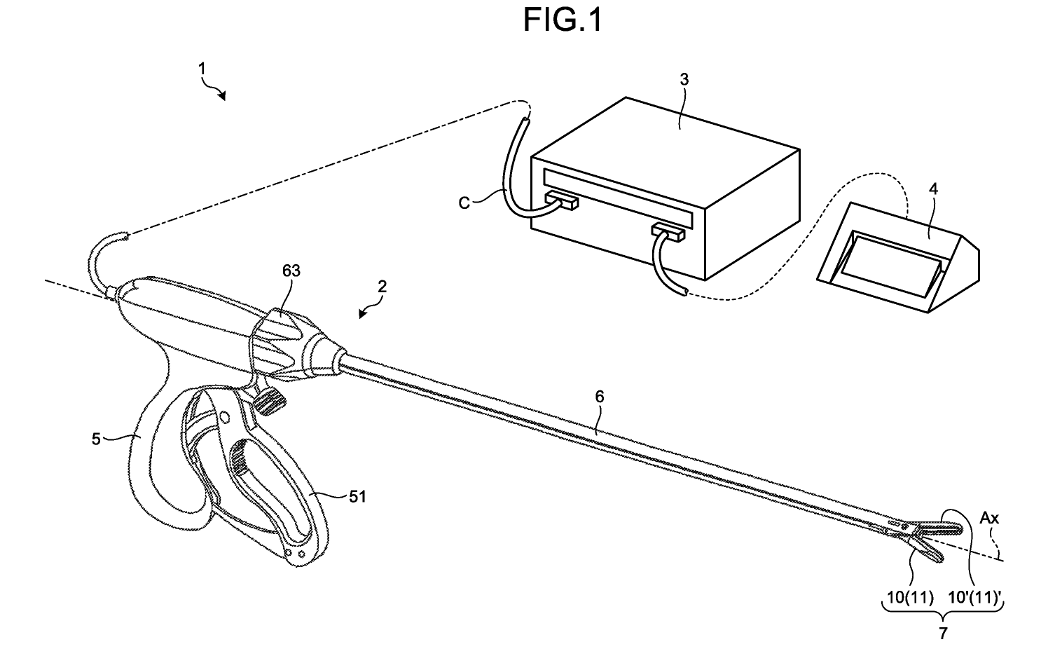

Schematic Configuration of Treatment System FIG. 1 is a diagram schematically illustrating a treatment system 1 according to a first embodiment.

The treatment system 1 treats a living tissue as a treatment target by applying thermal energy to the living tissue (examples of the treatment including bonding (or anastomosis) and dissection). As illustrated in FIG. 1, the treatment system 1 is provided with a treatment tool 2, a control device 3, and a foot switch 4.

Configuration of Treatment Tool

The treatment tool 2 is, for example, a linear-type surgical medical treatment tool for performing treatment on a living tissue through an abdominal wall. As illustrated in FIG. 1, the treatment tool 2 is provided with an operation handle 5, a shaft 6, and a gripping unit 7.

The operation handle 5 is a part manually held by an operator. As illustrated in FIG. 1, the operation handle 5 is provided with an operation knob 51 for opening/closing first and second jaws 11 and 11′ constituting the gripping unit 7.

In FIG. 1, a configuration and a shape indicated by a reference numeral to which “′” is not added are the same as a configuration and a shape indicated by a reference numeral to which “′” is added. The same applies to the following drawings.

Configuration of Shaft

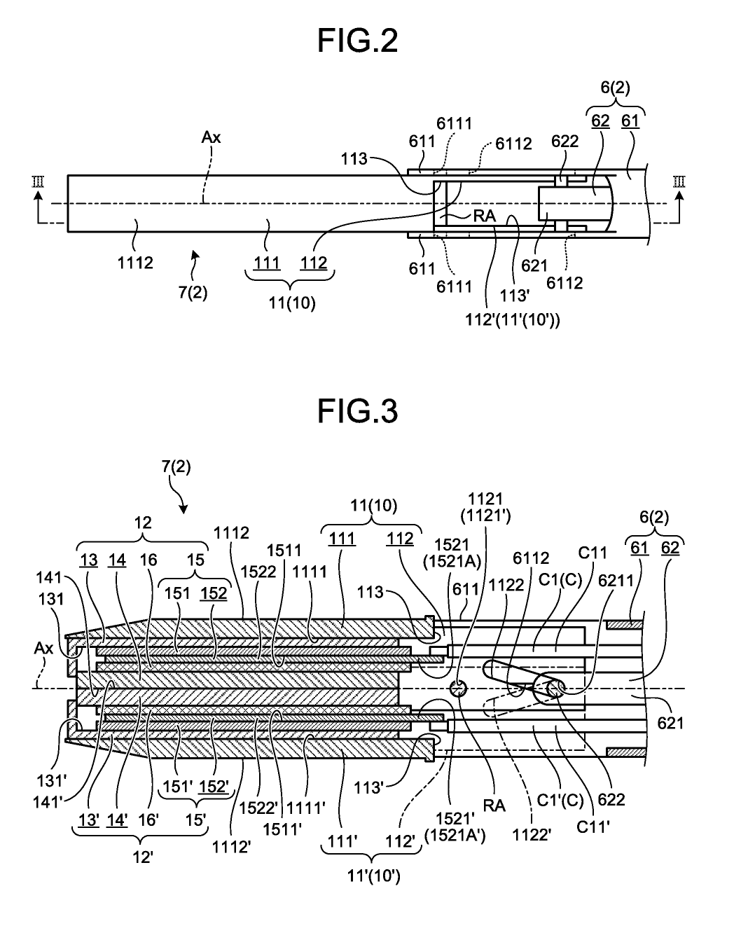

FIGS. 2 and 3 are diagrams illustrating the distal end part of the treatment tool 2. Specifically, FIG. 2 is a diagram in which the distal end part of the treatment tool 2 is viewed from the first jaw 11 side. In FIG. 2, illustration of first and second lead wires C1 and C1′ is omitted for convenience of description. FIG. 3 is a sectional view taken along line III-III of FIG. 2.

As illustrated in FIG. 1, the shaft 6 is an elongated member along a central axis Ax. One end of the shaft 6 is connected to the operation handle 5 via a rotation support member 63, and the other end of the shaft 6 pivotally supports the first and second jaws 11 and 11′ in an openable/closable manner. As illustrated in FIG. 2 or FIG. 3, the shaft 6 is provided with a tube section 61 and a rod 62.

The rotation support member 63 supports the shaft 6 and is attached to the operation handle 5 so as to be rotatable around the central axis Ax. In other words, by the rotation support member 63 being rotated in response to an operation by an operator, the shaft 6 and the first and second jaws 11 and 11′ attached to the shaft 6 rotate around the central axis Ax with the rotation support member 63.

The tube section 61 has a substantially cylindrical shape with one end of the tube section 61 connected to the rotation support member 63. On the other end side of the tube section 61, the first and second jaws 11 and 11′ are openably/closably supported.

In the tube section 61, an electric cable C (FIG. 1) connected to the control device 3 is disposed from one end side to the other end side via the operation handle 5 and the rotation support member 63. In FIG. 3, a pair of the first lead wires C1 and a pair of the second lead wires C1′ constituting the electric cable C are partially illustrated.

A pair of pivot support sections 611 is provided at the other end of the tube section 61. Each of the pivot support sections 611 protrudes toward the distal end of the treatment tool 2 (left side in FIGS. 2 and 3).

Each of the pair of pivot support sections 611 has an elongated and substantially flat plate shape. The pair of pivot support sections 611 is provided so that the pivot support sections 611 face each other in the upward-downward direction in FIG. 2 with a longitudinal direction along the central axis Ax.

The pair of pivot support sections 611 has the same shape. Therefore, the shape of one pivot support section 611 will be described below.

As illustrated in FIG. 2, in the pivot support section 611, a first bearing hole 6111 is formed closer to the distal end side (left side in FIG. 2) than the longitudinal middle position of the pivot support section 611. A rotary shaft RA is inserted into the first bearing hole 6111 through the front and back of the pivot support section 611.

In addition, a first track hole 6112 is formed in the pivot support section 611. As illustrated in FIG. 2 or FIG. 3, the first track hole 6112 is formed closer to the proximal end side (right side in FIGS. 2 and 3) than the first bearing hole 6111. The first track hole 6112 extends along the central axis Ax through the front and back of the pivot support section 611.

The rod 62 is disposed in the tube section 61 and moves forward and backward along the central axis Ax in response to an operation of the operation knob 51 by an operator. In other words, the rod 62 constitutes a part of an opening/closing system opening/closing the first and second jaws 11 and 11′. As illustrated in FIG. 2 or FIG. 3, the rod 62 is provided with a rod main body 621 and a shaft section 622.

An elongated bar-shaped member constitutes the rod main body 621. The rod main body 621 is a part that moves forward and backward along the central axis Ax in response to an operation of the operation knob 51 by an operator. An insertion hole 6211 is formed on the distal end side (left side in FIGS. 2 and 3) of the rod main body 621. The insertion hole 6211 penetrates the rod main body 621 in a direction orthogonal to the central axis Ax, and the shaft section 622 is inserted into the insertion hole 6211.

The shaft section 622 has a cylindrical shape and is inserted into the insertion hole 6211 of the rod main body 621. In a state where the shaft section 622 is inserted in the insertion hole 6211, both ends of the shaft section 622 protrude outwards from the rod main body 621 as illustrated in FIG. 2. The ends of the shaft section 622 protruding outwards from the rod main body 621 are inserted into the respective first track holes 6112 of the pair of pivot support sections 611 and second track holes 1122 and 1122′ of the first and second jaws 11 and 11′ (FIG. 3).

[Configuration of Gripping Unit]

The gripping unit 7 is a part gripping a living tissue to treat the living tissue. As illustrated in FIG. 2 or FIG. 3, the gripping unit 7 is provided with a first gripping unit 10 that has the first jaw 11 and a first energy applying structure 12 and a second gripping unit 10′ that has the second jaw 11′ and a second energy applying structure 12′.

The first and second jaws 11 and 11′ correspond to the pair of jaws according to the present disclosure.

Configuration of First Jaw

The first jaw 11 is a part rotatably and pivotally supported by the pair of pivot support sections 611 via the rotary shaft RA. As illustrated in FIG. 2 or FIG. 3, the first jaw 11 is provided with a jaw main body 111 and a jaw connecting section 112.

As illustrated in FIG. 2, the jaw main body 111 has an elongated and substantially flat plate shape with a width dimension (transverse length dimension) slightly smaller than the separation dimension of the pair of pivot support sections 611. One surface of the jaw main body 111 functions as a gripping surface 1111 (FIG. 3) to which the first energy applying structure 12 is attached. In the following description, the surface on the side that is opposite to the gripping surface 1111 will be referred to as a back surface 1112 (FIGS. 2 and 3).

The jaw connecting section 112 is a part connecting the first jaw 11 to the tube section 61. The jaw connecting section 112 has an elongated and substantially flat plate shape. The jaw connecting section 112 is integrally formed on the upper side in FIG. 2 on one end side (right side in FIGS. 2 and 3) of the jaw main body 111 in a state where the longitudinal direction of the jaw connecting section 112 is along the longitudinal direction of the jaw main body 111 and orthogonal to the jaw main body 111.

A second bearing hole 1121 is formed in the jaw connecting section 112 through the front and back of the jaw connecting section 112. As illustrated in FIG. 3, the second bearing hole 1121 is formed closer to the distal end side (left side in FIG. 3) than the longitudinal middle position of the jaw connecting section 112. In other words, the first jaw 11 is pivotally supported, so as to be rotatable around the rotary shaft RA, with respect to the tube section 61 (pair of pivot support sections 611) by abutting of the jaw connecting section 112 against the inner surface of the pivot support section 611, which is one of the pair of pivot support sections 611, and insertion of the rotary shaft RA into each of the first bearing hole 6111 and the second bearing hole 1121.

In addition, the second track hole 1122 is formed in the jaw connecting section 112. As illustrated in FIG. 3, the second track hole 1122 is formed closer to the proximal end side (right side in FIG. 3) than the second bearing hole 1121. The second track hole 1122 extends in a direction crossing the central axis Ax through the front and back of the jaw connecting section 112.

Specifically, the second track hole 1122 is inclined toward the second bearing hole 1121 and the upper side in FIG. 3. In the state illustrated in FIG. 3 (state where the first and second jaws 11 and 11′ are closed), the end section of the second track hole 1122 on the right side in FIG. 3 is set at the same height position as the first track hole 6112. In other words, in the state illustrated in FIG. 3, the height position of the second track hole 1122 gradually rises with respect to the first track hole 6112 toward the second bearing hole 1121. An end section of the shaft section 622 is inserted into the second track hole 1122.

In the following description, the L-shaped edge part constituted by the jaw main body 111 and the jaw connecting section 112 will be referred to as a notch section 113 (FIGS. 2 and 3) for convenience of description.

Configuration of First Energy Applying Structure

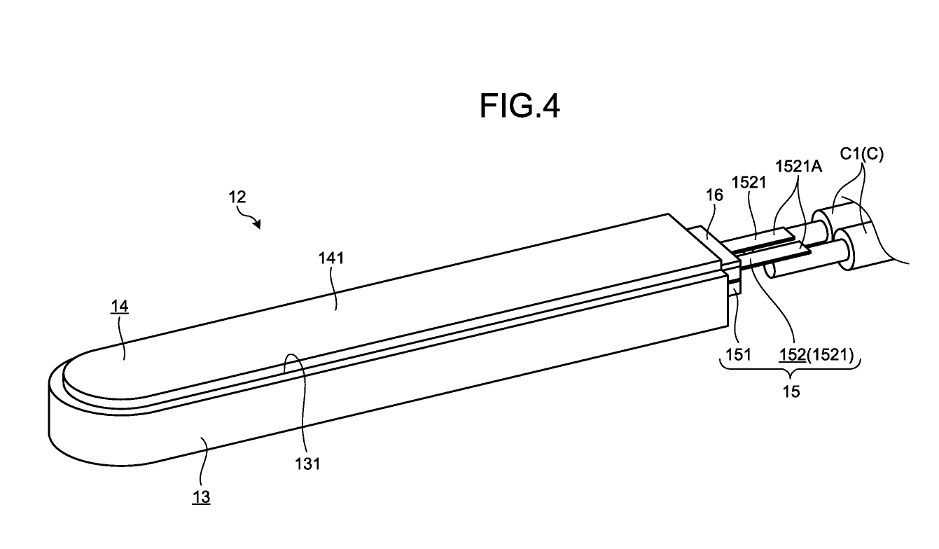

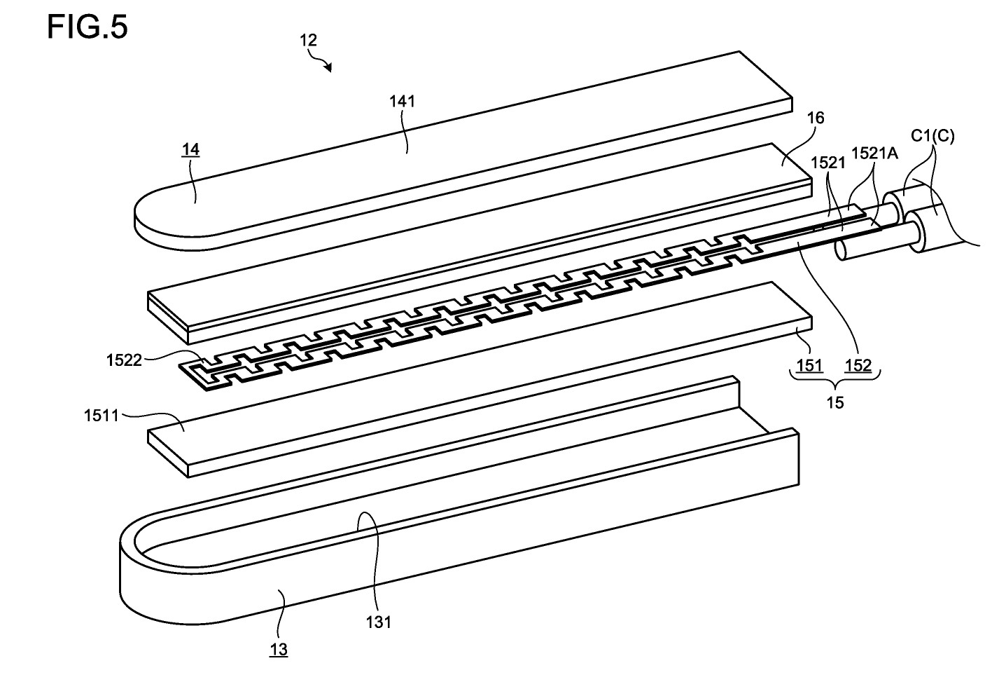

FIGS. 4 and 5 are diagrams illustrating the first energy applying structure 12. Specifically, FIG. 4 is a perspective view in which the first energy applying structure 12 is viewed from the side of a treatment surface 141 coming into contact with a living tissue. FIG. 5 is an exploded perspective view of FIG. 4.

As illustrated in FIG. 4 or FIG. 5, the first energy applying structure 12 is provided with a cover member 13, a heat transfer plate 14, a heater 15, an adhesive sheet 16, and the pair of first lead wires C1.

The cover member 13 has a substantially rectangular parallelepiped shape extending along the central axis Ax of the tube section 61. A recessed section 131 is provided at a substantially central width-direction position of the cover member 13, and the recessed section 131 extends from one end of the cover member 13 (right end section in FIGS. 4 and 5) toward the other end side along the longitudinal direction of the cover member 13.

The heat transfer plate 14, the heater 15, and the adhesive sheet 16 are installed in the recessed section 131 as illustrated in FIG. 4.

The cover member 13 described above is a molded resin material such as fluororesin.

The heat transfer plate 14 is made of a material such as copper. The heat transfer plate 14 is an elongated thin plate (elongated thin plate extending in the longitudinal direction of the cover member 13 (left-right direction in FIGS. 4 and 5)) extending from the distal end (left end section in FIG. 3) toward the proximal end (right end section in FIG. 3) of the first jaw 11 (gripping surface 1111). In a state where a living tissue is gripped by the gripping unit 7 (state where the first and second jaws 11 and 11′ are closed), the treatment surface 141 (surface on the upper side in FIGS. 4 and 5), which is the surface of the heat transfer plate 14, transfers heat from the heater 15 to the living tissue (applies thermal energy to the living tissue) in contact with the living tissue.

Here, the planar shape of the heat transfer plate 14 is set to be substantially identical to the planar shape of the recessed section 131.

The heater 15 has a heat-generating part. The heater 15 functions as a sheet heater heating the heat transfer plate 14 by means of the heat generation. As illustrated in FIG. 4 or FIG. 5, the heater 15 is provided with a substrate 151 and a wiring pattern 152.

The substrate 151 is made of polyimide as an insulating material. The substrate 151 is an elongated sheet (elongated sheet extending in the longitudinal direction of the cover member 13) extending from the distal end toward the proximal end of the first jaw 11 (gripping surface 111). The substrate 151 corresponds to the first substrate according to the present disclosure.

The material of the substrate 151 is not limited to polyimide. Alternatively, a highly heat-resistant insulating material such as aluminum nitride, alumina, glass, and zirconia may be adopted.

Here, the width dimension of the substrate 151 is set to be slightly smaller than the width dimension of the heat transfer plate 14. In addition, the length dimension (longitudinal length dimension) of the substrate 151 is set to be longer than the length dimension (longitudinal length dimension) of the heat transfer plate 14.

The substrate 151 may be made of a conductive material. In this case, insulating coating may be performed for electrical insulation from the wiring pattern 152.

The wiring pattern 152 corresponds to the first wiring pattern according to the present disclosure. The wiring pattern 152 is formed by processing of stainless steel (SUS 304) as a conductive material. As illustrated in FIG. 4 or FIG. 5, the wiring pattern 152 is provided with a pair of connecting sections 1521 and an electric resistance pattern 1522 (FIG. 5). The wiring pattern 152 is pasted by thermocompression bonding to one surface 1511 of the substrate 151 (FIG. 5).

The material of the wiring pattern 152 is not limited to stainless steel (SUS 304). Also adoptable are another stainless steel material (such as 400 series) and a conductive material such as platinum and tungsten.

As illustrated in FIG. 4 or FIG. 5, each of the pair of connecting sections 1521 extends along the longitudinal direction of the substrate 151 and the pair of connecting sections 1521 is provided to face each other along the width direction of the substrate 151. In a state where the wiring pattern 152 is pasted to the one surface 1511 of the substrate 151, each of the pair of connecting sections 1521 partially protrudes from the one surface 1511 to the right side in FIG. 5 along the in-plane direction of the one surface 1511. In the following description, the protruding parts of the pair of connecting sections 1521 will be referred to as overhang sections 1521A (FIGS. 4 and 5) for convenience of description.

One end of the electric resistance pattern 1522 is connected (electrically continuous) to one of the connecting sections 1521 and extends along a U-shape conforming to the outer edge shape of the substrate 151 while meandering in a wave shape from the end. The other end of the electric resistance pattern 1522 is connected (electrically continuous) to the other connecting section 1521.

The electric resistance pattern 1522 generates heat by a voltage being applied (by a current being carried) to the pair of connecting sections 1521 via the pair of first lead wires C1 by the control device 3.

As illustrated in FIG. 4 or FIG. 5, the adhesive sheet 16 is interposed between the heat transfer plate 14 and the heater 15. In a state where a part of the heater 15 protrudes from the heat transfer plate 14, the adhesive sheet 16 provides adhesive fixing between the one surface 1511 of the substrate 151 and the rear surface of the heat transfer plate 14 (surface on the side that is opposite to the treatment surface 141). This adhesive sheet 16 is an elongated sheet (elongated sheet extending in the longitudinal direction of the cover member 13) having good thermal conductivity and insulating properties, enduring a high temperature, and having adhesiveness. The adhesive sheet 16 is formed by mixing of a resin such as epoxy or polyurethane with a highly heat-conductive filler (nonconductive material) such as alumina, boron nitride, graphite, and aluminum nitride.

Here, the width dimension of the adhesive sheet 16 is set to be substantially equal to the width dimension of the substrate 151. In addition, the length dimension (longitudinal length dimension) of the adhesive sheet 16 is set to be longer than the length dimension (longitudinal length dimension) of the heat transfer plate 14 and shorter than the length dimension (longitudinal length dimension) of the substrate 151.

The heat transfer plate 14 is disposed to cover the entire region of the electric resistance pattern 1522. In addition, the adhesive sheet 16 is disposed to cover the entire region of the electric resistance pattern 1522 and cover a part of the pair of connecting sections 1521. In other words, the adhesive sheet 16 is disposed in a state of protruding to the right side in FIGS. 4 and 5 with respect to the heat transfer plate 14.

As a result of the above disposition, the proximal end side end section of the substrate 151 (right end section in FIGS. 3 to 5) is positioned closer to the proximal end side (right side in FIG. 3) of the first jaw 11 (gripping surface 1111) than the proximal end side end section of the heat transfer plate 14 (right end section in FIGS. 3 to 5).

Each of the pair of first lead wires C1 is a part that constitutes the electric cable C connected to the control device 3 and is to be a current-carrying path to the wiring pattern 152. The first lead wire C1 corresponds to the lead wire according to the present disclosure. As illustrated in FIG. 4 or FIG. 5, each of the pair of first lead wires C1 is positioned on a side (lower side in FIGS. 4 and 5) separated from the heat transfer plate 14 with respect to the wiring pattern 152 and the pair of first lead wires C1 is electrically connected (bonded) to the respective overhang sections 1521A of the pair of connecting sections 1521 from the separated side.

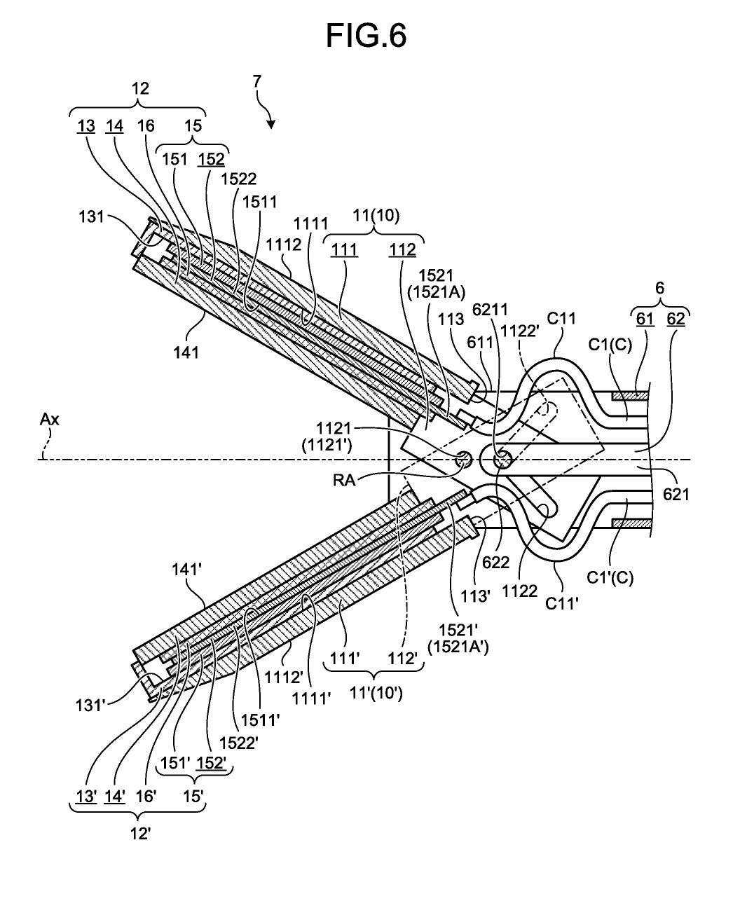

In the first embodiment, the first lead wire C1 has a curved section C11 (refer to FIGS. 3 and 6) shaped such that the central axis of the first lead wire C1 is convexly curved in a state where no force is applied in the direction in which the first lead wire C1 is pulled. In the state illustrated in FIG. 3, a pulling force is applied to the outside in the axial direction of the lead wire and along the axial direction with respect to the first lead wire C1 in a state where the first and second jaws 11 and 11′ are closed, and thus the curved section C11 is linearly deformed. The pair of first lead wires C1 is electrically connected to the respective overhang sections 1521A such that the curved section C11 is disposed at a position (position between the respective gripping surfaces 1111 and 1111′ of the first and second jaws 11 and 11′) protruding from the other end of the tube section 61 (end section on the left side in FIG. 3) and the direction in which the curved section C11 is curved (direction of the convexity thereof) is on the side separated from the second gripping unit 10′ (upper side in FIG. 3).

Configuration of Second Jaw

The second jaw 11′ has the same configuration and shape as the first jaw 11 and faces the first jaw 11. The second jaw 11′ is rotatably and pivotally supported by the pair of pivot support sections 611 via the rotary shaft RA in a posture in which the first jaw 11 is inverted.

Since the second jaw 11′ has the same configuration and shape as the first jaw 11, a reference numeral to which “′” is added is attached to a configuration common to the first jaw 11 and the second jaw 11′ so that description thereof is omitted.

Configuration of Second Energy Applying Structure

The second energy applying structure 12′ has the same configuration and shape as the first energy applying structure 12 and faces the first energy applying structure 12. The second energy applying structure 12′ is attached to the gripping surface 1111′ in a posture in which the first energy applying structure 12 is inverted.

Since the second energy applying structure 12′ has the same configuration and shape as the first energy applying structure 12, a reference numeral to which “′” is added is attached to a configuration common to the first energy applying structure 12 and the second energy applying structure 12′ so that description thereof is omitted.

Here, a substrate 151′ corresponds to the second substrate according to the present disclosure. A wiring pattern 152′ corresponds to the second wiring pattern according to the present disclosure.

In the first embodiment, each of the substrates 151 and 151′ is configured to be disposed between the first lead wire C1 and the second lead wire C1′ as illustrated in FIG. 3.

Opening/Closing Operation of First and Second Jaws

An opening/closing operation of the first and second jaws 11 and 11′ described above will be described below.

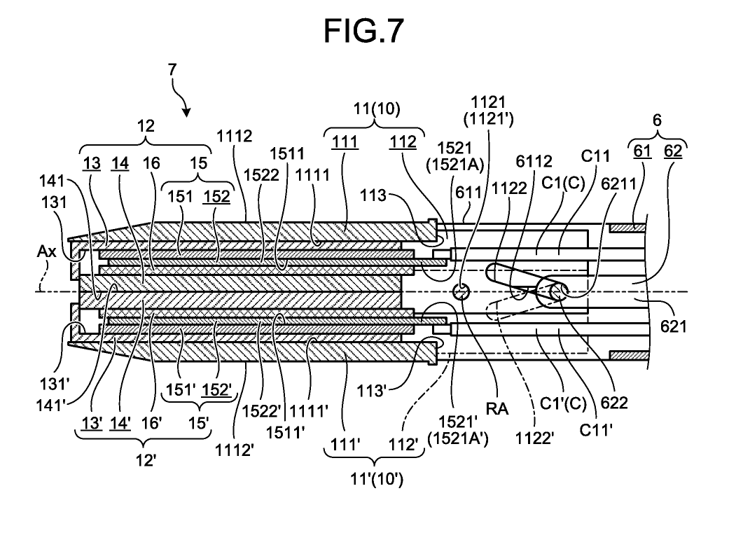

FIGS. 6 and 7 are diagrams for describing the opening/closing operation of the first and second jaws 11 and 11′. Specifically, FIG. 6 is a sectional view corresponding to FIG. 3 and illustrates a state where the first and second jaws 11 and 11′ are open. FIG. 7 is a sectional view corresponding to FIG. 3 and illustrates a state where the first and second jaws 11 and 11′ are closed.

Illustrated in FIG. 6 is a state where an operator is yet to operate the operation knob 51. In this state, the first and second jaws 11 and 11′ are open as illustrated in FIG. 6.

Once the operation knob 51 is operated by an operator from the “open state” illustrated in FIG. 6, the rod 62 moves to the operation handle 5 side (right side in FIGS. 6 and 7). As a result of the movement of the rod 62, the shaft section 622 moves from the left side toward the right side in FIG. 6 or FIG. 7 in each of the first track holes 6112 and each of the second track holes 1122 and 1122′.

As described above, each first track hole 6112 provided in the tube section 61 is set so as to extend along the central axis Ax. On the other hand, as described above, the second track hole 1122 provided in the first jaw 11 is set such that the height position thereof gradually rises with respect to each first track hole 6112 toward the left side in FIG. 6 or FIG. 7. In addition, the second jaw 11′ is inverted in posture with respect to the first jaw 11. Therefore, the second track hole 1122′ provided in the second jaw 11′ has a height position gradually falling with respect to each first track hole 6112 toward the left side in FIG. 6 or FIG. 7.

Accordingly, during the movement from the left side toward the right side in FIG. 6 or FIG. 7 in each of the first track holes 6112 and each of the second track holes 1122 and 1122′, the shaft section 622 moves while pressing the edge part of each of the second track holes 1122 and 1122′. Then, the first and second jaws 11 and 11′ rotate around the rotary shaft RA in the direction in which the first and second energy applying structures 12 and 12′ approach each other. Finally, the “closed state” illustrated in FIG. 7 is achieved.

In the “closed state” illustrated in FIG. 7, each of the pair of first lead wires C1 and the pair of second lead wires C1′ is pulled to the outside in the axial direction of the lead wires (in the left-right direction in FIG. 7) along the axial direction to be given a substantially linear shape. In other words, each of the curved sections C11 and C11′ is positioned in the space between the first and second jaws 11 and 11′ (does not protrude outwards from the first and second jaws 11 and 11′).

Once the operation of the operation knob 51 is released by an operator from the “closed state” illustrated in FIG. 7, the rod 62 moves from the right side toward the left side in FIG. 6 or FIG. 7 conversely to the above. As the rod 62 moves, the first and second jaws 11 and 11′ rotate around the rotary shaft RA in the direction in which the first and second energy applying structures 12 and 12′ are separated from each other conversely to the above. Finally, the “open state” illustrated in FIG. 6 is achieved.

In the “open state” illustrated in FIG. 6, the pair of first lead wires C1 and the pair of second lead wires C1′ are not in a state of being pulled to the outside in the axial direction of the lead wires. Accordingly, each of the curved sections C11 and C11′ is convexly curved in the direction of returning to the original state thereof, and the curved sections C11 and C11′ protrude to the outside of the first and second jaws 11 and 11′ from the back surfaces 1112 and 1112′ via the notch sections 113 and 113′ of the first and second jaws 11 and 11′, respectively.

Configurations of Control Device and Foot Switch

The foot switch 4 is a part operated with an operator's foot. Depending on the operation on the foot switch 4, carrying of a current to the heaters 15 and 15′ (electric resistance patterns 1522 and 1522′) is switched ON or OFF.

Means for the ON-OFF switching is not limited to the foot switch 4. Alternatively, a manual operation switch or the like may be adopted.

The control device 3 is configured to include a central processing unit (CPU) and the like and comprehensively controls the operation of the treatment tool 2 in accordance with a predetermined control program. More specifically, in accordance with an operation on the foot switch 4 by an operator (ON operation for carrying a current), the control device 3 heats the heat transfer plates 14 and 14′ by applying a voltage to the heaters 15 and 15′ (electric resistance patterns 1522 and 1522′) via the pair of first lead wires C1 and the pair of second lead wires C1′ (by carrying a current to each of the electric resistance patterns 1522 and 1522′), respectively.

Operation of Treatment System

An operation of the treatment system 1 described above will be described below.

An operator manually holds the treatment tool 2 and inserts the distal end part of the treatment tool 2 (a part of the gripping unit 7 and a part of the shaft 6) into an abdominal cavity through an abdominal wall by using, for example, a trocar. Then, the operator grips a living tissue of a treatment target with the gripping unit 7 by operating the operation knob 51.

Next, the operator operates the foot switch 4 to switch ON carrying of a current from the control device 3 to the treatment tool 2 (heaters 15 and 15′). As a result of the ON switching, the control device 3 applies a voltage to the electric resistance patterns 1522 and 1522′ via the pair of first lead wires C1 and the pair of second lead wires C1′, respectively. Then, the heat transfer plates 14 and 14′ are heated. Then, by the heat of the heat transfer plates 14 and 14′, the living tissue gripped by the heat transfer plates 14 and 14′ is treated.

In the treatment tool 2 according to the first embodiment described above, each of the pair of first lead wires C1 is positioned on the side separated from the heat transfer plate 14 with respect to the wiring pattern 152 and the pair of first lead wires C1 is electrically connected to the pair of connecting sections 1521 (respective overhang sections 1521A) from the separated side, respectively. The second energy applying structure 12′ has the same configuration and shape as the first energy applying structure 12 and is attached to the gripping surface 1111′ in a posture in which the first energy applying structure 12 is inverted.

Accordingly, the pair of first lead wires C1 and the pair of second lead wires C1′ constituting the first and second energy applying structures 12 and 12′ do not mechanically interfere with each other in a state where the first and second jaws 11 and 11′ are closed. In addition, a configuration for widening the gap between the first and second jaws 11 and 11′ in order to avoid the mechanical interference is unnecessary.

Accordingly, the treatment tool 2 according to the first embodiment is effective in that the diameter of the living tissue gripping unit 7 may be reduced.

In particular, the pair of first lead wires C1 is directly connected to the respective overhang sections 1521A of the pair of connecting sections 1521.

Accordingly, neither a configuration nor a shape needs to be devised for the substrate 151 (second and third embodiments to be described later) in adopting a configuration for connecting the pair of first lead wires C1 to the pair of connecting sections 1521 from the side separated from the heat transfer plate 14 with respect to the wiring pattern 152. Therefore, the structure of the substrate 151 may be simplified. The same applies to the second energy applying structure 12′ (substrate 151′).

In the treatment tool 2 according to the first embodiment, the pair of first lead wires C1 has the curved section C11 in which the central axis of the first lead wire C1 is curved. In a state where the first and second jaws 11 and 11′ are closed, the curved section C11 is positioned in the space between the first and second jaws 11 and 11′ (does not protrude outwards from the first and second jaws 11 and 11′). The same applies to the pair of second lead wires C1′.

Accordingly, when the distal end part of the treatment tool 2 is inserted into an abdominal cavity, a reduction in the diameter of the distal end part is not inhibited by the part of the pair of first lead wires C1 (pair of second lead wires C1′) protruding to the outside of the first and second jaws 11 and 11′. In other words, the distal end part of the treatment tool 2 may be smoothly inserted into an abdominal cavity.

In a state where the first and second jaws 11 and 11′ are closed, each of the pair of first lead wires C1 and the pair of second lead wires C1′ is pulled to the outside in the axial direction of the lead wires along the axial direction. In a state where the first and second jaws 11 and 11′ are open, a force for pushing to the shaft 6 side acts on the pair of first lead wires C1 and the pair of second lead wires C1′. Therefore, the pair of first lead wires C1 and the pair of second lead wires C1′ are curved in response to the force. In a case where a configuration for curving in the tube section 61 is adopted in view of the curving, the diameter dimension of the tube section 61 needs to be increased, and then the diameter of the shaft 6 may not be reduced.

In the first embodiment, in contrast, the curved section C11 protrudes to the outside of the first and second jaws 11 and 11′ from the back surface 1112 of the first jaw 11 via the notch section 113 in a state where the first and second jaws 11 and 11′ are open. The same applies to the pair of second lead wires C1′. In other words, in a state where the first and second jaws 11 and 11′ are open, not a configuration curved in the tube section 61 but a configuration protruding to the outside of the first and second jaws 11 and 11′ is adopted.

Therefore, it is not necessary to increase the diameter dimension of the tube section 61 and the diameter of the shaft 6 may be reduced.

In a state where the first and second jaws 11 and 11′ are open and the curved sections C11 and C11 are curved, a force acts in the direction in which each pair of overhang sections 1521A is pressed from the pair of first lead wires C1 and the pair of second lead wires C1′. In other words, no force acts in the direction in which the pair of first lead wires C1 and the pair of second lead wires C1′ are separated from each pair of overhang sections 1521A, and thus separation may be prevented between the pair of first lead wires C1 and the pair of second lead wires C1′ and each pair of overhang sections 1521A.

For a configuration to be adopted in which the pair of first lead wires C1 are directly connected to the pair of connecting sections 1521 (respective overhang sections 1521A), the length dimension (longitudinal length dimension) of the substrate 151 needs to be shorter than the length dimension (longitudinal length dimension) of the wiring pattern 152. The adhesive sheet 16 and the wiring pattern 152 are thin materials. Accordingly, bending stress concentration occurs in the adhesive sheet 16 and the wiring pattern 152 around the proximal end side end section of the heat transfer plate 14.

In the first embodiment, the proximal end side end section of the substrate 151 is positioned closer to the proximal end side of the first jaw 11 (gripping surface 1111) than the proximal end side end section of the heat transfer plate 14 for prevention of bending stress concentration in the vicinity of the proximal end side end section of the heat transfer plate 14 (adhesive sheet 16 and wiring pattern 152). This configuration results in bending stress generation around the proximal end side end section of the substrate 151, and thus bending stress concentration in the vicinity of the proximal end side end section of the heat transfer plate 14 (adhesive sheet 16 and wiring pattern 152), which is relatively vulnerable to bending stress, may be avoided. The same applies to the second energy applying structure 12′.

### Second Embodiment

A second embodiment will be described below.

In the following description, the same reference numerals are used to designate the same configurations as those of the above-described first embodiment, and detailed description thereof is omitted or simplified.

The treatment tool according to the second embodiment differs from the treatment tool 2 according to the above-described first embodiment in terms of the configurations of the heaters 15 and 15′. Accordingly, only the configuration of the heater according to the second embodiment will be described below.

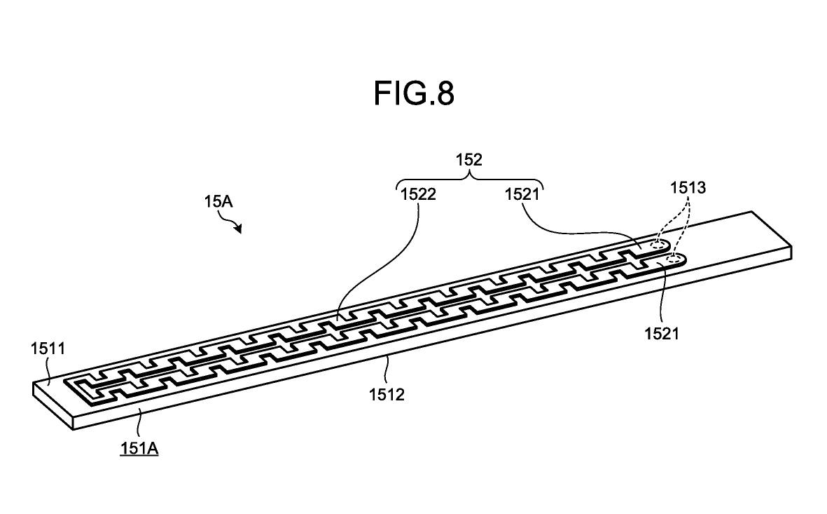

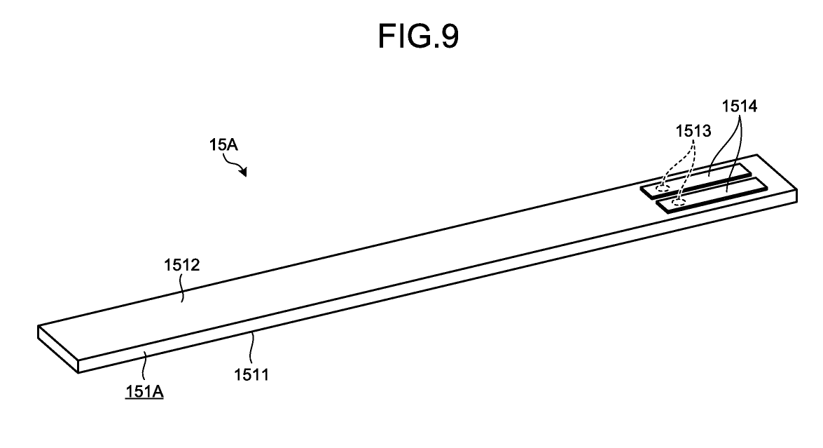

FIGS. 8 and 9 are diagrams illustrating a heater 15A according to the second embodiment. Specifically, FIG. 8 is a perspective view in which the heater 15A is viewed from the one surface 1511 side of a substrate 151A. FIG. 9 is a perspective view in which the heater 15A is viewed from the other surface 1512 side on the side that is opposite to the one surface 1511.

In the heater 15A, the substrate 151A that differs from the substrate 151 is adopted with respect to the heater 15 described in the first embodiment.

With respect to the substrate 151 described in the first embodiment, the substrate 151A is set to have a lengthened length dimension (longitudinal length dimension). More specifically, the length dimension (longitudinal length dimension) of the substrate 151A is set to be longer than the length dimension (longitudinal length dimension) of the wiring pattern 152 as illustrated in FIG. 8.

At each of the positions in the substrate 151A where the pair of connecting sections 1521 is disposed, a through hole 1513 penetrating the one surface 1511 and the other surface 1512 is formed as illustrated in FIG. 8 or FIG. 9.

In the substrate 151A, a pair of electrodes 1514 electrically connected to the through hole 1513 is respectively provided on the other surface 1512 as illustrated in FIG. 9.

In the second embodiment, the pair of first lead wires C1 and the pair of second lead wires C1′ are electrically connected (bonded) with respect to the pair of electrodes 1514, respectively.

As is the case with the first embodiment described above, in the second embodiment, the proximal end side end section of the substrate 151A (right end section in FIGS. 8 and 9) is positioned closer to the proximal end side of the gripping surface 1111 (1111′) than the proximal end side end section of the heat transfer plate 14 (14′).

Effects similar to those of the first embodiment described above are achieved even in a case where the heater 15A according to the second embodiment described above is adopted.

The wiring pattern 152 according to the present embodiment is not limited to a configuration for thermocompression bonding-based pasting to the one surface 1511 of the substrate 151A. Also adoptable is a configuration for vapor deposition-based formation on the one surface 1511.

### Third Embodiment

A third embodiment will be described below.

In the following description, the same reference numerals are used to designate the same configurations as those of the above-described first embodiment, and detailed description thereof is omitted or simplified.

The treatment tool according to the third embodiment differs from the treatment tool 2 according to the above-described first embodiment in terms of the configurations of the heaters 15 and 15′. Accordingly, only the configuration of the heater according to the third embodiment will be described below.

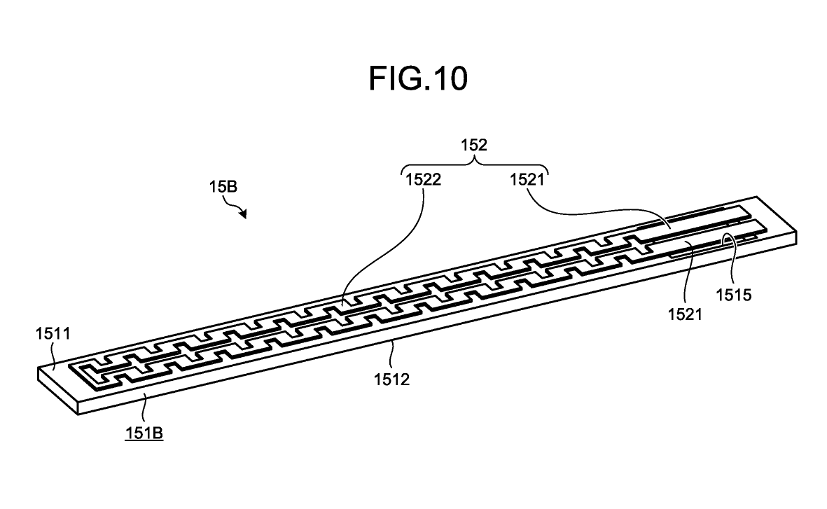

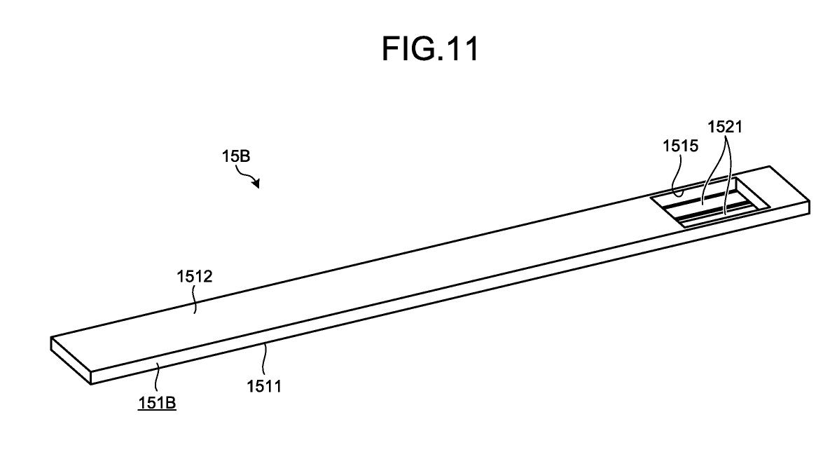

FIGS. 10 and 11 are diagrams illustrating a heater 15B according to the third embodiment. Specifically, FIG. 10 is a perspective view in which the heater 15B is viewed from the one surface 1511 side of a substrate 151B. FIG. 11 is a perspective view in which the heater 15B is viewed from the other surface 1512 side.

In the heater 15B, the substrate 151B that differs from the substrate 151 is adopted with respect to the heater 15 described in the first embodiment.

With respect to the substrate 151 described in the first embodiment, the substrate 151B is set to have a lengthened length dimension (longitudinal length dimension). More specifically, the length dimension (longitudinal length dimension) of the substrate 151B is set to be longer than the length dimension (longitudinal length dimension) of the wiring pattern 152 as illustrated in FIG. 10.

At the positions in the substrate 151B where the pair of connecting sections 1521 are disposed, an aperture section 1515 penetrating the one surface 1511 and the other surface 1512 is formed as illustrated in FIG. 10 or FIG. 11.

In the second embodiment, the pair of first lead wires C1 and the pair of second lead wires C1′ are electrically connected (bonded) with respect to the pair of connecting sections 1521 via the aperture section 1515 from the other surface 1512 side, respectively.

As is the case with the first embodiment described above, in the third embodiment, the proximal end side end section of the substrate 151B (right end section in FIGS. 10 and 11) is positioned closer to the proximal end side of the gripping surface 1111 (1111′) than the proximal end side end section of the heat transfer plate 14 (14′). The aperture section 1515 is positioned closer to the proximal end side of the gripping surface 1111 (1111′) than the proximal end side end section of the heat transfer plate 14 (14′).

Effects similar to those of the first embodiment described above are achieved even in a case where the heater 15B according to the third embodiment described above is adopted.

### Fourth Embodiment

A fourth embodiment will be described below.

In the following description, the same reference numerals are used to designate the same configurations as those of the above-described first embodiment, and detailed description thereof is omitted or simplified.

With respect to the treatment tool 2 according to the first embodiment described above, the treatment tool according to the fourth embodiment adopts high-frequency energy as well as thermal energy as the energy that is applied to a living tissue.

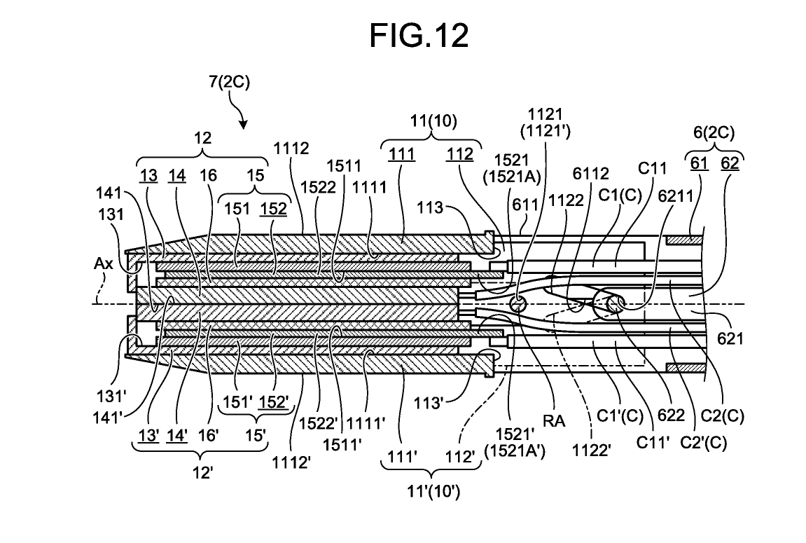

FIG. 12 is a diagram illustrating a treatment tool 2C according to the fourth embodiment. Specifically, FIG. 12 is a diagram corresponding to FIG. 3.

As illustrated in FIG. 12, third lead wires C2 and C2′ are added to the treatment tool 2C according to the fourth embodiment with respect to the treatment tool 2 (FIG. 3) according to the first embodiment described above.

The third lead wires C2 and C2′ constitute the electric cable C connected to the control device 3. As illustrated in FIG. 12, the third lead wires C2 and C2′ are electrically connected (bonded) to the heat transfer plates 14 and 14′, respectively. High-frequency electric power is supplied to the heat transfer plates 14 and 14′ via the third lead wires C2 and C2′ by the control device 3. As the high-frequency electric power is supplied, a high-frequency current flows between the heat transfer plates 14 and 14′ and Joule heat is generated in the living tissue gripped between the heat transfer plates 14 and 14′. The living tissue is treated as a result of the Joule heat generation.

In other words, the heat transfer plates 14 and 14′ have a function as the electrode according to the present disclosure. The third lead wires C2 and C2′ serve as paths for carrying the high-frequency electric power to the heat transfer plates 14 and 14′.

Effects similar to those of the first embodiment described above are achieved even in a case where the treatment tool 2C, which applies high-frequency energy as well as thermal energy to a living tissue, is adopted as in the fourth embodiment described above.

### Other Embodiments

Although modes for carrying out the present disclosure have been described so far, the present disclosure is limited by the first to fourth embodiments described above.

In the first to fourth embodiments described above, a configuration is adopted in which thermal energy is applied to a living tissue from both of the first and second gripping units 10 and 10′ (first and second energy applying structures 12 and 12′). The present disclosure is not limited thereto. For example, one of the first and second energy applying structures 12 and 12′ is omitted. In other words, a configuration may be adopted in which thermal energy is applied to a living tissue from the other of the first and second energy applying structures 12 and 12′ alone.

In the first to fourth embodiments described above, another system may be adopted as the opening/closing system opening/closing the first and second jaws 11 and 11′ without having to be limited to the opening/closing system described in the first to fourth embodiments.

In the first to fourth embodiments described above, thermal energy and high-frequency energy are adopted as the energy to be applied to a living tissue. Alternatively, a configuration may be adopted in which ultrasonic energy is applied to a living tissue in addition to thermal energy.

The treatment tool according to the present disclosure is effective in that the diameter of a living tissue-gripping structure may be reduced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a pair of openable/closable jaws;
a substrate provided on at least one of the pair of jaws;
a wiring pattern provided on one surface of the substrate in a state of being electrically insulated from the substrate, the wiring pattern including
an electric resistance pattern configured to generate heat when a current is carried to the electric resistance pattern, and
a connecting section configured to be electrically continuous to the electric resistance pattern; and
a heat transfer plate disposed facing the one surface and configured to transfer, to a living tissue, the heat from the electric resistance pattern in contact with the living tissue; and
a lead wire configured to be a current-carrying path to the wiring pattern, the lead wire being positioned on a side separated from the heat transfer plate with respect to the wiring pattern and being electrically connected to the connecting section from the separated side.

2. The treatment tool according to claim 1, further comprising a heat-transferring adhesive sheet that is interposed between the substrate and the heat transfer plate, the heat-transferring adhesive sheet being configured to cover an entire region of the electric resistance pattern, and adhere and fix the substrate and the heat transfer plate.

3. The treatment tool according to claim 1, wherein
the wiring pattern is provided on the one surface in a state where at least a part of the connecting section protrudes outwards from the one surface along an in-plane direction of the one surface, and
the lead wire is electrically connected to the part of the connecting section protruding outwards from the one surface.

4. The treatment tool according to claim 2, wherein
the wiring pattern is provided on the one surface in a state where at least a part of the connecting section protrudes outwards from the one surface along an in-plane direction of the one surface, and
the lead wire is electrically connected to the part of the connecting section protruding outwards from the one surface.

5. The treatment tool according to claim 1, wherein
the wiring pattern is provided in the one surface,
a through hole configured to be electrically continuous to the connecting section and penetrate the one surface and an other surface on a side opposite to the one surface is provided to the substrate, and
the lead wire is electrically connected to the connecting section via the through hole.

6. The treatment tool according to claim 2, wherein
the wiring pattern is provided in the one surface,
a through hole configured to be electrically continuous to the connecting section and penetrate the one surface and an other surface on a side opposite to the one surface is provided to the substrate, and
the lead wire is electrically connected to the connecting section via the through hole.

7. The treatment tool according to claim 1, wherein
the wiring pattern is provided in the one surface,
an aperture section penetrating the one surface and the other surface on a side opposite to the one surface is provided at a position in the substrate where the connecting section is disposed, and
the lead wire is electrically connected to the connecting section via the aperture section.

8. The treatment tool according to claim 2, wherein
the wiring pattern is provided in the one surface,
an aperture section penetrating the one surface and the other surface on a side opposite to the one surface is provided at a position in the substrate where the connecting section is disposed, and
the lead wire is electrically connected to the connecting section via the aperture section.

9. The treatment tool according to claim 7, wherein
each of the heat transfer plate and the substrate extends from a distal end of the jaw toward a proximal end of the jaw, and
the aperture section is positioned closer to the proximal end side than an end section of the heat transfer plate on the proximal end side.

10. The treatment tool according to claim 8, wherein
each of the heat transfer plate and the substrate extends from a distal end of the jaw toward a proximal end of the jaw, and
the aperture section is positioned closer to the proximal end side than an end section of the heat transfer plate on the proximal end side.

11. The treatment tool according to claim 1, wherein
each of the heat transfer plate and the substrate extends from a distal end of the jaw toward the proximal end of the jaw, and
an end section of the substrate on the proximal end side is positioned closer to the proximal end side than the end section of the heat transfer plate on the proximal end side.

12. The treatment tool according to claim 1, wherein
the substrate includes
a first substrate provided on one of the pair of jaws, and
a second substrate provided on an other jaw of the pair of jaws,
the wiring pattern includes
a first wiring pattern provided on the first substrate, and
a second wiring pattern provided on the second substrate,
the lead wire includes
a first lead wire configured to be a current-carrying path to the first wiring pattern, and
a second lead wire configured to be a current-carrying path to the second wiring pattern, and
the first substrate and the second substrate are disposed between the first lead wire and the second lead wire.

13. The treatment tool according to claim 1, further comprising:
an electrode provided on at least one of the pair of jaws; and
a third lead wire configured to be a path for carrying high-frequency electric power to the electrode.

14. The treatment tool according to claim 8, further comprising:
an electrode provided on at least one of the pair of jaws; and
a third lead wire configured to be a path for carrying high-frequency electric power to the electrode.

15. The treatment tool according to claim 1, further comprising a tubular shaft configured to pivotally support the pair of jaws in an openable/closable manner on one end side, wherein
each of the pair of jaws includes a gripping surface gripping the living tissue,
the lead wire is inserted into the shaft, the lead wire being disposed in a state of protruding between the pair of jaws from one end side of the shaft, and having a curved section in which a central axis of the lead wire is curved at the protruding part,
the substrate is provided on one of the pair of jaws,
the one jaw is provided with a notch section notched in a direction along a central axis of the shaft from an end section of the shaft on one end side, and
the curved section is positioned closer to the other jaw side than a back surface of the one jaw on a side opposite to the gripping surface in a state where the pair of jaws is closed, and
the curved section protrudes to an outside of the one jaw from the back surface via the notch section in a state where the pair of jaws is open.
